(19) United States
(12) Patent Application Publication
(10) Pub. No.: US 2025/0177507 A1
(43) Pub. Date: Jun. 5, 2025

(54) Multivalent Vaccine Compositions

(71) Applicant: Inventprise, Inc, Redmond, WA (US)

(72) Inventors: Subhash V. Kapre, Redmond, WA (US); Manoj Kumar, Redmond, WA (US)

(73) Assignee: Inventprise, Inc., Redmond, WA (US)

(21) Appl. No.: 18/967,715

(22) Filed: Dec. 4, 2024

Related U.S. Application Data
(60) Provisional application No. 63/606,439, filed on Dec. 5, 2023.

(51) Int. Cl. A61K 39/108; A61K 39/00

(52) U.S. Cl. CPC A61K 39/0266; A61K 2039/54; A61K 2039/55; A61K 2039/55505; A61K 2039/55555; A61K 2039/55572; A61K 2039/55577; A61K 2039/6037; A61K 2039/6068; A61K 2039/627; A61K 2039/70

(57) ABSTRACT

The invention is directed to compositions and methods for the prevention and treatment of infections where the causative agent is a Klebsiella microorganism. In particular, the invention is directed to multivalent immunogenic compositions and vaccines containing multiple different serotypes of Klebsiella capsular polysaccharides and/or subcapsular polysaccharides that are prevalent in low- and middle-income countries where the burden of disease is high.

### REFERENCE TO RELATED APPLICATIONS

This application claims priority to U.S. Provisional Application No. 63/606,439, filed Dec. 5, 2023, the entirety of which is incorporated by reference.

### BACKGROUND

### 1. Field of the Invention

This invention is directed to compositions and methods for the prevention and treatment of infections where the causative agent is a species of Klebsiella. In particular, the invention is directed to immunogenic compositions containing multiple antigenic serotypes of Klebsiella which are representative of serotypes that are prevalent in low- and middle-income countries.

### 2. Description of the Background

Klebsiella pneumoniae is a Gram-negative bacterium and considered to be a clinical-superbug that causes diverse, difficult to treat infections due in part to its variable capsular polysaccharides (i.e. antigens). The K. pneumoniae microorganism colonizes mucous membranes, causing serious infections making K. pneumoniae a leading cause of hospital acquired infections (HAI). Infection results in symptoms such as pneumonia, meningitis, wound infections, and sepsis. Widespread antibiotic resistance in K. pneumoniae either through intrinsic mechanisms or via acquisition from different species, especially in hospital environments, limits the therapeutic options against this pathogen, further aggravating the disease burden. Klebsiella infections have been associated with 789,903 deaths globally in 2019, and accounts for 81% of antimicrobial resistance (AMR) associated deaths. Data from facilities in CHAMPS and the Burden of Antibiotic Resistance in Neonates from Developing Societies study identified K. pneumoniae as the primary pathogen in early (lesser than 72 h) and late (greater than 72 h) onset neonatal sepsis across all geographies, accounting for 30% of neonatal sepsis deaths. There are 9 serologically defined sub-capsular O types, and 77 capsular K types.

Klebsiella infections can be prevented by vaccination. One of the approaches of vaccine development against K. pneumoniae is a conjugate vaccine using conjugates of capsular (K) and sub-capsular (O) antigens representing the important serotypes. Although formulations based on capsular polysaccharides have been proposed, the high variability in capsular serotypes limits vaccine coverage. Many vaccine candidates have been proposed, some of which have reached clinical trials. Different other strategies including whole cell vaccines, outer membrane vesicles (OMVs), ribosome, polysaccharide, lipopolysaccharide (LPS), and protein-based formulations have also been attempted. To date, there are no commercially available vaccines against K. pneumoniae infection.

### SUMMARY OF THE INVENTION

The present invention overcomes the problems and disadvantages associated with current strategies and designs and provides new compositions and methods of treating and preventing infections attributed to multiple serotypes of Klebsiella.

One embodiment of the invention is directed to an immunogenic composition containing at least five different polysaccharide serotypes of K. pneumoniae, said serotypes selected from the group consisting of serotypes of capsular (K) polysaccharides K2, K25, K62, K102, K149, K15, K30, K17, K23, K64, K54, K10, K122, K14, K3, and K24, and sub-capsular (O) polysaccharides O1, O2afg, O3b, O4, O5, O2a, and O3a. Preferably the composition comprises at least 10 different serotypes of K. pneumoniae, at least 15 different serotypes of K. pneumoniae, at least 20 different serotypes of K. pneumoniae, or at least 23 or more different serotypes of K. pneumoniae. Preferred serotype combinations include, for example, K. pneumoniae serotypes K2, K25, K62, K102, K149, O1, O2afg, O3b, O4, and O5, or K. pneumoniae serotypes K2, K25, K102, O1, and O5. These serotypes are found to be prevalent in in most geographical areas of the world and especially in low- and middle-income countries. Preferably the one or more of the polysaccharides are from about 10 kDA to about 1,000 kDa in molecular weight.

Preferably, one or more of the polysaccharides are conjugated to one or more carrier proteins. The one or more polysaccharides may be conjugated to one or more carrier proteins through mono-functional, bi-functional, and/or multifunctional spacer/linkers. Preferably two or more polysaccharides are conjugated to a single carrier protein via a bi-functional or multifunctional spacer/linker Preferred carrier proteins include, but are not limited to native or recombinant cross-reactive material (CRM) or domain of CRM, CRM197, tetanus toxin, tetanus toxin heavy chain proteins, diphtheria toxoid, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, Shigella invasion plasmid antigen B (IpaB), native or recombinant Cholera toxin B subunit, OmpA, Fimbriae protein, K. pneumoniae enterotoxin and modifications thereof. Composition preferably contain a polysaccharide to protein ratio of from about 0.2 to about 3.0, and the total amount of K or O polysaccharides for each serotype conjugate are from about 1 μg to about 25 μg per human dose. Preferably, the total amount of K or O polysaccharides is 2 μg or less per dose, 4 μg or less per dose, 6 μg or less per dose, 8 μg or less per dose, 10 μg or less per dose, 12 μg or less per dose, 15 μg or less per dose, or 20 μg or less per dose.

Another embodiment of the invention is directed to a vaccine comprising the immunogenic composition disclosed here. Preferably the vaccine further comprises a pharmaceutically acceptable carrier and/or an adjuvant. Preferred pharmaceutically acceptable carriers include, but are not limited to oil, water, water-in-oil or oil-in-water mixtures, 20 mM phosphate buffered saline, aluminum phosphate, an alcohol, a buffer, a mono-, di- or polysaccharide, a sugar alcohol, sucrose, maltose, lactose, sorbitol, mannitol, trehalose, a glycerol, an amino acid, histidine, glycine, arginine, a preservative, a stabilizer, a surfactant, polysorbate, and combinations thereof. Preferred adjuvants include, but are not limited to aluminum salts, calcium phosphate, monophosphoryl lipid A (MPLA), a liposome of MPLA, a recombinant alternative of MPLA, saponin QS-21, a TLR7/8 agonist, and derivatives and combinations thereof.

Vaccines as disclosed herein may exist as liquid formulations, lyophilized formulations, or a combination wherein some serotypes exist as a lyophilized formulation and others as a liquid formulation. Such combinations may be combined before administration and/or the lyophilized formulation rehydrated before combination and administration to a mammal. Preferably the moisture content of a lyophilized vaccine is not more than about 5%, not more than about 4%, not more than about 3%, not more than about 2%, or not more than about 1%, and/or the vaccine has a pH of from about 5 to about 8, more preferable from about 5.6 or more to about 7.6 or less.

Another embodiment of the invention is directed to the manufacture of the immunogenic compositions and vaccines disclosed herein. Manufacturing comprises: activating each of the K or O polysaccharide serotypes; conjugating the activated K or O polysaccharide serotypes directly or indirectly through linkers to carrier proteins; and isolating the immunogenic composition wherein the linkers are bi-functional spacer/linkers and/or multifunctional spacer/linkers. Preferably the linkers are PEG-linkers. Also preferably, isolating comprises removal of endotoxin that may be present via multimodal chromatography.

Another embodiment of the invention is directed to treating or preventing an infection of a Klebsiella microorganism by administering the immunogenic composition or vaccine as disclosed here to a mammal. Preferably administration is parenteral or dermal via microneedles.

Other embodiments and advantages of the invention are set forth in part in the description, which follows, and in part, may be obvious from this description, or may be learned from the practice of the invention.

### DESCRIPTION OF THE INVENTION

The present invention overcomes the problems and disadvantages associated with current strategies and designs and provides new compositions, tools and methods for the prevention and treatment of infections wherein the causative agent is Klebsiella.

One embodiment of the invention is directed to multivalent immunogenic compositions containing at least 5 K. pneumoniae serotypes representing capsular (K) polysaccharides K2, K25, K62, K102, K149, K15, K30, K17, K23, K64, K54, K10, K122, K14, K3, and K24, or sub-capsular (O) polysaccharides of O1, O2afg, O3b, O4, O5, O2a, and O3a. Preferably the number of serotypes per multivalent composition is at least ten, at least 15, at least 20, or at least 23 or more. A preferred composition contains K. pneumoniae serotypes K2, K25, K62, K102, K149, O1, O2afg, O3b, O4, and O5. Another preferred composition contains K. pneumoniae serotypes K2, K25, K102, O1, and O5. Preferably one or more of the polysaccharides, or all, are from about 10 kDA to about 1,000 kDa in molecular weight.

Preferably the K and O polysaccharides are conjugated or otherwise coupled to carrier proteins directly or indirectly through a linker molecule. Conjugation may involve but is not limited to cyanylation chemistry and reductive amination chemistry. Linker are preferably bivalent or multivalent linkers and may be PEG-linkers. Preferably each polysaccharide of the composition is conjugated to a single carrier protein through a PEG-ylated hydrazide linker. Alternatively, multiple polysaccharides may be conjugated to a single carrier protein. The conjugation method may involves activation of the saccharide with CDAP to form a cyanate ester. Similarly, carrier protein can be derivatized with a PEGylated hydrazide or any other bi-functional group containing linker using EDC/sNHS chemistry. The activated saccharide can be coupled via a spacer (linker) group to a preferably hydrazide group on the carrier protein. Preferably, the polysaccharide cyanate ester is coupled with PEG-ylated hydrazide (HZ-PEG-HZ)) to the carrier protein using carbodiimide (e.g., EDAC or EDC) chemistry via a Hydrazide group on the protein carrier. Similarly, polysaccharide can be oxidized to create-aldehyde (—CHO) or carboxylic acid (—COOH) group, and then conjugated with carrier protein either directly or through a linker using reductive amination or EDC/sNHS chemistry.

Preferably the carrier proteins are selected from the group consisting of native or recombinant cross-reactive material (CRM) or domain of CRM, CRM197, tetanus toxin, tetanus toxin heavy chain proteins, diphtheria toxoid, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, Shigella invasion plasmid antigen B (IpaB), native or recombinant Cholera toxin B subunit, OmpA, Fimbriae protein, K. pneumoniae enterotoxin and modifications thereof.

After conjugation of the capsular polysaccharide to the carrier protein, the polysaccharide-protein conjugates can be purified (e.g., enriched with respect to the amount of polysaccharide-protein conjugate) by a concentration/diafiltration operations, and depth filtration. After the individual glycoconjugates are purified, they can be compounded to formulate the immunogenic composition of the disclosure, which can be used as a vaccine.

Another embodiment of the invention is directed to vaccines comprising the immunogenic compositions as disclosed herein. Preferably such immunogenic compositions and vaccines contain a pharmaceutically acceptable diluent, excipient, or carrier such as, for example, water or a saline solution. Other pharmaceutically acceptable carriers include, for example, oil, water, water-in-oil or oil-in-water mixtures, an alcohol, a buffer, a mono-, di- or poly-saccharide, a sugar alcohol, a glycerol, or a combination thereof. The composition may contain ingredients such as a buffer, an amino acid such as, for example, histidine, arginine, a preservative, or a stabilizer, polysorbate, an adjuvant such as an aluminum phosphate, and/or a lyophilization excipient. Also preferably such immunogenic compositions and vaccines contain an adjuvant. Preferred adjuvants include, for example, aluminum based adjuvants, Freund's, a liposome, saponin, lipid A, squalene, and derivatives and combinations thereof. Preferred adjuvants include, for example, aluminum phosphate, aluminum hydroxide, aluminum hydroxyphosphate, AS01 (Adjuvant System 01) which is a liposome-based adjuvant which comprises QS-21 (a saponin fraction extracted from Quillaja saponaria Molina), and 3-O-desacyl-4′-monophosphoryl lipid A (MPL; a non-toxic derivative of the lipopolysaccharide from Salmonella minnesota) and on occasion a ligand such as a toll-like receptor (e.g., TLR4), AS01b which is a component of the adjuvant Shingrix, ALF (Army Liposome Formulation) which comprises liposomes containing saturated phospholipids, cholesterol, and/or monophosphoryl lipid A (MPLA) as an immunostimulant. ALF has a safety and a strong potency. AS01 is included in the malaria vaccine RTS,S (MOSQUIRIX®). ALF modifications and derivatives include, for example, ALF adsorbed to aluminum hydroxide (ALFA), ALF containing QS21 saponin (ALFQ), and ALFQ adsorbed to aluminum hydroxide (ALFQA). A preferred adjuvant formulation comprises Freund's adjuvant, a liposome, saponin, lipid A, squalene, unilamellar liposomes having a liposome bilayer that comprises at least one phosphatidylcholine (PC) and/or phosphatidylglycerol (PG), as phospholipids, which may be dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and/or distearyl phosphatidylglycerol (DSPG), a cholesterol, a monophosphoryl lipid A (MPLA), and a saponin.

Preferably the immunogenic compositions and vaccines disclosed herein do not require cold storage and may be maintained at ambient temperatures from manufacture to administration to subjects in need. Immunogenic compositions and vaccines as disclosed herein may be maintained as liquids, in a liquid formulation, or lyophilized to a powder and rehydrated before use. Also, vaccines may comprise a combination of liquid and powder forms that are separately maintained wherein some serotypes are in a lyophilized formulation and others in a liquid formulation. Before administration the forms are combined or the powder form is rehydrated and mixed with the liquid form. Preferably the moisture content of the lyophilized vaccine component is not more than 3%. Also preferably, the vaccine has a pH of from about 5 to about 8, or more preferably, from about 5.6 to about 7.6.

Another embodiment of the invention is directed to methods for the treatment and/or prevention of K. pneumoniae infections comprising administering the immunogenic compositions or vaccines disclosed herein to a subject. Administration may be by any conventional route which is used in the field of vaccines, in particular by the systemic administration such as via parenteral route (e.g., intravenously, intramuscularly, subcutaneously, intradermal), or may be via a non-parental route (e.g., oral such as via pills, capsules, syrups, or topical such as ointments, patches). Preferably, the route of administration is dermal via microneedles. Preferably there is no side effects, such as pain, tissue damage, infection, fever, fussiness, drowsiness, headache, loss of appetite, and/or vomiting as a consequence of administration. Preferably, dermal administration does not produce any more than minimal or no swelling, soreness, or redness at the site of administration which, if present, preferably lasts no more than about 24 hours or less to about 48 hours or less. An effective amount of the composition comprises a dose needed to elicit antibodies that significantly reduce the likelihood or severity of infection. Preferably the immune response generated is humoral and cellular and induces phagocytosis and/or killing of the microorganisms of the infection.

Preferably the subject is a mammal, such as a human, or another animal including but not limited to bovine, canine, equine, porcine, and avian species. Preferably the composition produces an immunogenic response in the mammal upon administration. The amount of the immunogenic composition(s) and the time needed for the administration of such immunogenic composition(s) will be within the purview of the ordinary-skilled artisan having benefit of the present teachings.

The administration of a therapeutically-effective, pharmaceutically-effective, and/or prophylactically-effective amount of the disclosed immunogenic compositions may be achieved by a single administration. Alternatively, in some circumstances, it may be desirable to provide multiple, or successive administrations of the immunogenic compositions, either over a relatively short, or even a relatively prolonged period of time, as may be determined by the skilled person overseeing the administration of such compositions. An effective dose comprises amounts in the range of about 0.1 μg to about 1 mg total protein or target antigen per animal or mammal. In one exemplary embodiment, the vaccine dosage range is about 0.1 μg to about 10 mg per mammal. However, one may prefer to adjust dosage based on the amount of antigen delivered. In either case, these ranges are merely guidelines from which one of ordinary skill in the art may deviate according to conventional dosing techniques. Precise dosages may be determined by assessing the immunogenicity of the conjugate produced in the appropriate host so that an immunologically effective dose is delivered. An immunologically effective dose is one that stimulates the immune system of the mammal to establish an immune response to the immunogenic composition or vaccine. Preferably, a level of immunological memory sufficient to provide long-term protection against disease caused by microbial infection is obtained. Preferably, vaccinations provide protection against a pathogenic infection for more than a one-year cycle. More preferably, protection is provided for up to 2 years, 5 years, 10 years, 15 years, 20 years, or longer.

Another embodiment of the invention is directed to methods for the manufacture of the immunogenic compositions disclosed herein. One preferred method comprises: activating each of the K or O polysaccharide serotypes; conjugating the activated K or O polysaccharide serotypes directly or indirectly through linkers to carrier proteins; and isolating the immunogenic composition wherein the linkers are bi-functional spacer/linkers and/or multifunctional spacer/linkers. Preferably the linkers are PEG-linkers. Also preferably, isolating comprises removal of possible endotoxin that may be present via multimodal chromatography using high capacity, endotoxin removal resin.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. It is intended that the specification and examples be considered exemplary only with the true scope and spirit of the invention indicated by the following claims. Furthermore, the term “comprising of” includes the terms “consisting of” and “consisting essentially of.”

1. An immunogenic composition containing at least five different polysaccharide serotypes of K. pneumoniae, said serotypes selected from the group consisting of serotypes of capsular (K) polysaccharides K2, K25, K62, K102, K149, K15, K30, K17, K23, K64, K54, K10, K122, K14, K3, and K24, and sub-capsular (O) polysaccharides O1, O2afg, O3b, O4, O5, O2a, and O3a.

2. The composition of claim 1, which comprises at least 10 different serotypes of K. pneumoniae.

3. The composition of claim 1, which comprises at least 15 different serotypes of K. pneumoniae.

4. The composition of claim 1, which comprises at least 20 different serotypes of K. pneumoniae.

5. The composition of claim 1, which comprises at least 23 different serotypes of K. pneumoniae.

6. The composition of claim 1, which contains K. pneumoniae serotypes K2, K25, K62, K102, K149, O1, O2afg, O3b, O4, and O5.

7. The composition of claim 1, which contains K. pneumoniae serotypes K2, K25, K102, O1, and O5.

8. The composition of claim 1, wherein one or more of the polysaccharides are from about 10 kDA to about 1,000 kDa in molecular weight.

9. The composition of claim 1, wherein the one or more of the polysaccharides are conjugated to one or more carrier proteins.

10. The composition of claim 1, wherein the one or more polysaccharides are conjugated to one or more carrier proteins through mono-functional, bi-functional, and/or multifunctional spacer/linkers.

11. The composition of claim 1, wherein the two or more polysaccharides are conjugated to a single carrier protein via a bi-functional or multifunctional spacer/linker.

12. The composition of claim 1, wherein the one or more carrier proteins are selected from the group consisting of native or recombinant cross-reactive material (CRM) or domain of CRM, CRM197, tetanus toxin, tetanus toxin heavy chain proteins, diphtheria toxoid, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, Shigella invasion plasmid antigen B (IpaB), native or recombinant Cholera toxin B subunit, OmpA, Fimbriae protein, K. pneumoniae enterotoxin and modifications thereof.

13. The composition of claim 1, which has a polysaccharide to protein ratio of from about 0.2 to about 3.0.

14. The composition of claim 1, wherein the total amount of K or O polysaccharides is from about 1 μg to about 25 μg per dose.

15. A vaccine comprising the composition of claim 1.

16. The vaccine of claim 15, comprising a pharmaceutically acceptable carrier and/or an adjuvant.

17. The vaccine of claim 16, wherein the pharmaceutically acceptable carrier is selected from the group consisting of oil, water, water-in-oil or oil-in-water mixtures, phosphate buffered saline, aluminum phosphate, an alcohol, a buffer, a mono-, di- or polysaccharide, a sugar alcohol, sucrose, maltose, lactose, sorbitol, mannitol, trehalose, a glycerol, an amino acid, histidine, glycine, arginine, a preservative, a stabilizer, a surfactant, polysorbate, and combinations thereof.

18. The vaccine of claim 16, wherein the adjuvant is selected from the group consisting of aluminum salt, calcium phosphate, a liposome of monophosphoryl lipid A (MPLA), saponin QS-21, a TLR7/8 agonist, and derivatives and combinations thereof.

19. The vaccine of claim 15, which is a liquid formulation, a lyophilized formulation or a combination wherein some serotypes are in a lyophilized formulation and others in a liquid formulation.

20. The vaccine or claim 15, which has a moisture content of lyophilized component is not more than 3%.

21. The vaccine of claim 15, which has a pH of from about 5 to about 8.

22. The vaccine of claim 21, which has a pH of from about 5.6 to about 7.6.

23. A method for manufacture of the immunogenic composition of claim 1, comprising:
activating each of the K or O polysaccharide serotypes;
conjugating the activated K or O polysaccharide serotypes directly or indirectly through linkers to carrier proteins; and
isolating the immunogenic composition wherein the linkers are mono-functional spacer/linkers, bi-functional spacer/linkers and/or multifunctional spacer/linkers.

24. The method of claim 23, wherein the spacer/linkers are selected from but not limited to PEG-linkers and/or Hydrazine linkers.

25. The method of claim 23, wherein endotoxin that is present is removed via multimodal chromatography.

26. A method for treating or preventing an infection of a Klebsiella microorganism by administering the composition of claim 1 to a mammal.

27. The method of claim 26, wherein administration is parenteral.

28. A method for treating or preventing a K. pneumoniae infection comprising administering the composition of claim 1 to a subject.

29. The method of claim 28, wherein administration is parenteral.

30. The method of claim 29, wherein parenteral administration produces minimal or no soreness, redness, or swelling at the site of administration.
